(19) United States
(12) Patent Application Publication
(10) Pub. No.: US 2014/0338677 A1
(43) Pub. Date: Nov. 20, 2014

(54) FACIAL PROTECTION AND THERMOREGULATION FOR PATIENTS UNDERGOING SURGERY

(71) Applicant: MICHAEL DIMITRI SPARKUHL, Santa Paula, CA (US)

(72) Inventor: MICHAEL DIMITRI SPARKUHL, Santa Paula, CA (US)

(21) Appl. No.: 14/278,276

(22) Filed: May 15, 2014

Related U.S. Application Data
(60) Provisional application No. 61/824,744, filed on May 17, 2013.

(51) Int. Cl. A61B 19/00

(52) U.S. Cl. CPC A61B 19/40; A61B 2019/4036; A61B 2019/409; A61B 2019/4063
USPC 128/857

(57) ABSTRACT

A mask suitable for protecting the face of a patient during surgery comprises a transparent plastic shield which includes a transverse cutout that provides access to the nose and mouth, and a vertical gap which runs from the cutout to the shield's perimeter. Padding affixed to the perimeter of the shield's underside provides a cushion between the shield and face. The padding is discontinuous, enabling the mask to be installed or removed without dislodging tubing inserted into the patient's nose or mouth via the transverse cutout. An attachment mechanism secures the shield to the face. A warming hood may be coupled to the protective mask. The hood preferably comprises outer and inner layers, with an access point at which air can be introduced between the layers; perforations on the inner layer permit warm air to be dispersed onto the patient.

### RELATED APPLICATIONS

This application claims the benefit of provisional patent application No. 61/824,744 to Michael Dimitri Sparkuhl, filed May 17, 2013.

### BACKGROUND OF THE INVENTION

1. Field of the Invention

The present invention relates generally to facial protection and thermoregulation for patients undergoing surgery.

2. Description of the Related Art

There is a growing interest in the safety of patients undergoing surgery, to avoid errors, injuries, and complications. Because many adverse events are preventable, the Joint Commission of Hospitals, the major hospital certifying agency for Medicare and CMS, and the World Health Organization, have devoted considerable resources to educating and monitoring health care professionals in matters of patient safety and the prevention of surgery errors, injuries, and complications.

There are generally accepted and standardized approaches to patient positioning, and the use of padding, restraints, straps, bolsters, and various support mechanisms to prevent injuries. Methods of preventing injuries to the face during surgery vary depending on patient positioning. For example, there is considerable discussion of facial protection in anesthesia literature, which discusses patients in the face down or prone position when undergoing spinal surgery or other procedures requiring that position. The emphasis here is on eye, nose, and lip protection and airway access, with eye and airway having the highest priority and being at the greatest risk. There are a few products available to help protect against injuries in this position, such as contoured foam rubber pads, modified head gear pads, adhesive eye goggles, and table mirrors which allow an anesthesiologist to see a patient's face in the prone position. Some of these methods also apply to patients placed in the decubitus position (patient lying on right or left side).

The majority of operations, however, are performed with the patient in the supine or face up position. In this position the face is exposed and unprotected, rendering it vulnerable to falling or dropped objects, instruments inadvertently striking the face, eyes or teeth, unanticipated drips of chemicals, body fluids, prep-solutions, etc. Eye and tooth injuries are the most common facial injuries in the operating room, occurring in about 1:1000 cases. Considering that about 24-44 million operations are performed annually in the USA, there are many injuries occurring which probably go unreported.

Despite these concerns, facial protection of a patient in the supine position is not standardized; it is typically left up to the anesthesiologist to best determine how to protect the face from mechanical or fluid-related injury. Conventional protection efforts may involve, for example, periodic visual inspection so as to keep the surgical drapes, as well as the surgeon's hands, arms, instruments and instrument trays away from the patient's face. Folded sheets, towels, or foam rubber pads may also be placed over the face. However, using these approaches, an injury might occur while the anesthesiologist is otherwise occupied and not constantly watching the patient's face. In addition, access to the face required by the anesthesiologist may be compromised by the various non-transparent items placed on the face; those items might also provide inadequate facial protection. There are no known devices specifically designed for facial protection under general anesthesia when the patient is in a supine position.

Another problem that can arise during a surgical procedure is inadvertent intraoperative hypothermia, due to exposure of the head and face with comcomitant heat loss. This is known to be dangerous for the patient and can lead to surgical complications. It is known that the head loses as much as 30% of body heat under stress and anesthesia. Forced air heating units are commonly used for the extremities and torso, but none are independently available and dedicated to the head & neck area.

### SUMMARY OF THE INVENTION

A protective mask suitable for protecting the face of a patient during surgery is presented, which provides for predictable, standardized facial protection of patients under general anesthesia in the supine position. A warming hood is also presented which prevents heat loss from the head, neck, and shoulders, and may also be used to actively warm these areas to prevent inadvertent intraoperative hypothermia.

The present protective mask comprises a transparent plastic shield sized and shaped to protect the face of a patient when the mask is installed. The shield has a convex topside and an underside, and includes a transverse cutout through the shield which provides access to the patient's nose and mouth, and a vertical gap which runs from the transverse cutout to the lower perimeter of the shield. The transverse cutout is sized to permit the passage of tubing to the patient's nose and/or mouth.

Padding is affixed to the perimeter of the underside of the shield which contacts the patient's face and provides a cushion between the shield and the face when the mask is installed. The padding is arranged to allow the patient's face to remain substantially visible through the shield when the protective mask is installed. The padding is preferably pre-slit to be discontinuous in the area of the vertical gap, which enables the mask to be installed on or removed from the face of the patient without dislodging tubing inserted into the patient's nose or mouth. An attachment mechanism secures the shield and padding to the patient's face.

A warming hood may also be employed; the hood is arranged to be coupled to the protective mask and covers the head, neck and shoulders when so coupled. The hood preferably comprises a blanket having an outer layer and an inner layer, with the outer and inner layers heat sealed to each other at their respective perimeters. The blanket has at least one access point at which air can be introduced between the outer and inner layers; the inner layer comprises a plurality of micro-perforations through which air introduced via the access points is dispersed onto the patient.

These and other features, aspects, and advantages of the present invention will become better understood with reference to the following description and claims.

### BRIEF DESCRIPTION OF THE DRAWINGS

FIGS. 1a and 1b are perspective views of a protective mask per the present invention.

FIG. 1c is a side elevation view of a protective mask per the present invention.

FIG. 2 is a perspective view of the present protective mask, used in combination with a warming hood per the present invention.

### DETAILED DESCRIPTION OF THE INVENTION

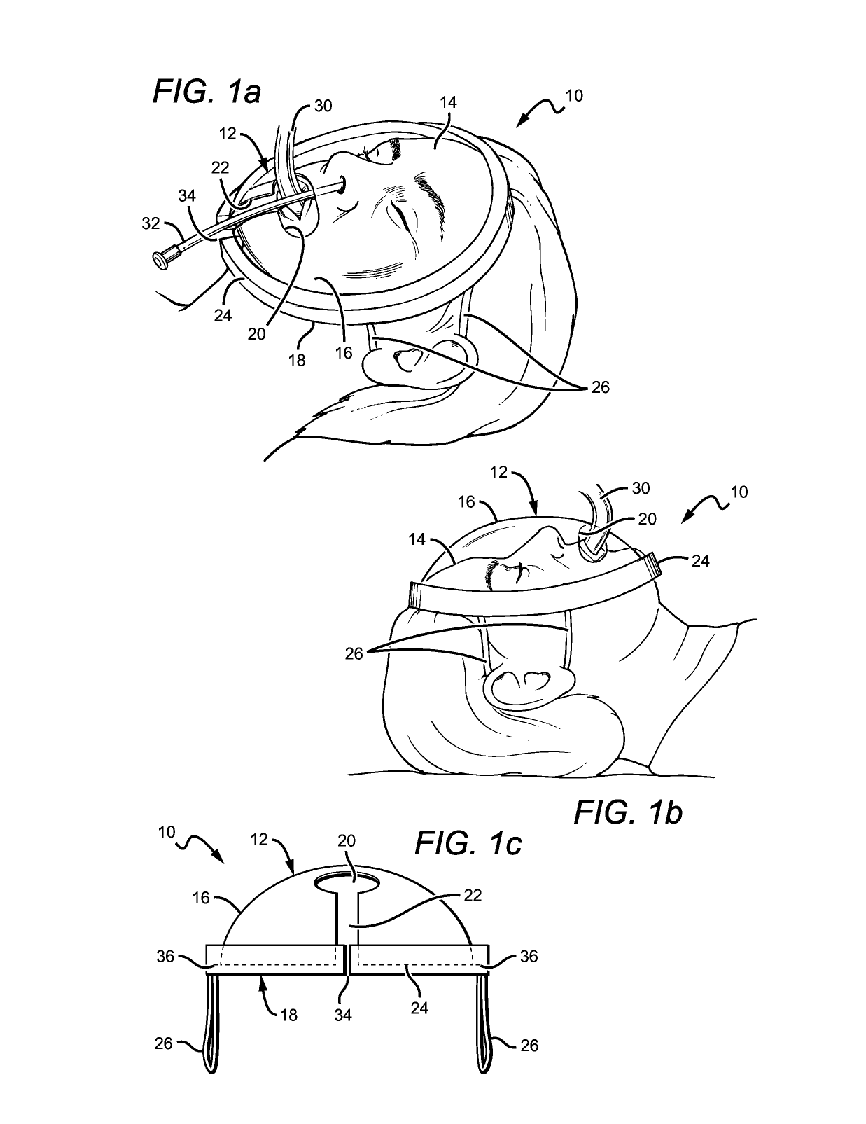

A protective mask, suitable for protecting the face of a patient during surgery, is shown in FIGS. 1a and 1b (perspective views) and 1c (front elevation view). The mask 10 comprises a transparent plastic shield 12 sized and shaped to protect the face of a patient 14 when the mask is installed on the patient's face. Shield 12 has a convex topside 16 and an underside 18. The shield includes a transverse cutout 20 through the shield which provides access to the patient's nose and mouth, and a vertical gap 22 which runs from the transverse cutout to the perimeter of the shield. The protective mask also includes padding 24 affixed to the perimeter of the underside of shield 12, which contacts the patient's face and provides a cushion between the shield and face when the mask is installed on the patient's head. Padding 24 is arranged to allow the patient's face to remain substantially visible through the shield when the mask is installed on the patient's face. An attachment mechanism 26 secures shield 12 and padding 24 to the face of patient 14.

Transverse cutout 20 is sized to permit the passage of tubing to the nose and mouth; this may include, for example, at least two of an endotracheal tube 30, a laryngeal mask airway tube (not shown), and a nasogastric tube 32. Mask 10 is arranged such that, when installed on the face of a patient, transverse cutout 20 is over the patient's mouth and vertical gap 22 runs from the transverse cutout to the perimeter of the shield over the patient's chin, such that the transverse cutout and vertical gap form a T-shaped opening through the shield.

Padding 24 includes a discontinuity 34 in the area of vertical gap 22. Plastic shield 12, transverse cutout 20, vertical gap 22 and discontinuous padding 24 are arranged such that the protective mask can be installed on or removed from the face of patient 14 without dislodging tubing (30, 32) inserted into the patient's nose or mouth. Transverse cutout 20 is suitably approximately 4 inches wide by 1 inch high and vertical gap 22 is suitably approximately 0.75 inch wide by 3 inches high, though both of these may be differently sized as needed for a given application. The vertical gap allows unencumbered rapid placement, positioning and removal of the mask with no tube disturbance or disconnect, while the transverse cutout allows further tube positioning from side to side, permitting flexibility of tube extension placement from the anesthesia machine.

Shield 12 is preferably a molded plastic shield made from FDA-approved medical grade plastic. In addition to protecting the face from injury due to falling objects or fluids, the shield may be arranged to protect the face—including the eyes—from ultraviolet rays, X-rays, and/or laser light. The shield is preferably fabricated by vacuforming plastic over a mold, with the tube cutouts made with a computer controlled cutter or die cutting before die-cutting the periphery of extraneous plastic to free the final transparent mask.

Padding 24 is preferably FDA-approved, hypoallergenic medical grade foam padding. The padding is preferably pre-slit prior to its being installed on shield 12 to provide discontinuity 34 in the area of vertical gap 22. Note that padding 24 may be implemented with materials other than foam padding. For example, the padding may be a flexible plastic bladder containing air or a conformable material, or comprise disposable cotton padding.

Attachment mechanism 26 is preferably affixed to plastic shield 12, though attachment to padding 24 is also contemplated. Attachment mechanism 26 can take a number of different forms; a preferred attachment mechanism comprises elastic ear loop retainers as illustrated in FIGS. 1a-1c. Alternatives include a behind-head elastic strap, a behind-head hook-and-loop strap, or a four point head strap harness typically used for anesthesia masks.

Another possible attachment mechanism is an adhesive between the patient's face and the surface of padding 24 which contacts the face. A preferred adhesive is an electron beam cure synthetic polymer.

Padding 24 could be attached to the shield in many different ways. In a preferred embodiment, plastic shield 12 includes a flange 36 which extends circumferentially approximately ¼″ from the perimeter of the shield (shown in FIG. 1c), so that it substantially forms a right angle with the topside of the shield. Padding 24 may then comprise first and second pieces of foam padding which are sandwiched on either side of flange 36 and affixed to the flange with double-sided sticky tape or other easily applied non-toxic adhesive. The ends of attachment mechanism 26 which are affixed to plastic shield 12 would typically pass through the sandwiched foam padding en route to the shield.

When attachment mechanism 26 comprises elastic ear loop retainers, the retainers are preferably pre-cut into thin elastic loops of an appropriate length, and attached with heat sealing or non-toxic glue to the right and left sides of plastic shield 12 to match generally accepted adult ear level.

Another possible means of attaching padding 24 to shield 12 requires inserting the shield into a circumferential slit cut into the padding; assuming foam padding, the slit might be created using, for example, a foam cutting knife set up in a jig. Once inserted into the slit, the shield could be held in place with a non-toxic rapid-drying adhesive, or heat sealed. The padding might be die-cut, or molded into the desired shape using injection molding techniques.

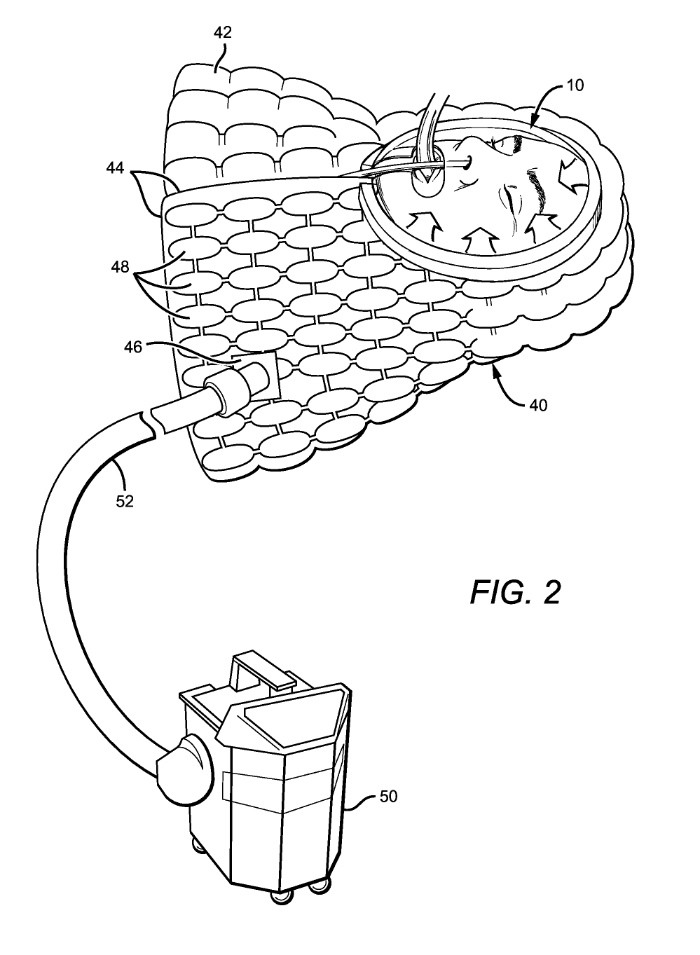

A warming hood can be coupled to the protective mask, which covers the head, neck and shoulders when so coupled. An exemplary embodiment is shown in FIG. 2, with warming hood 40 coupled to a protective mask 10 as described above. The hood 40 preferably comprises a blanket, which is preferably coupled to mask 10 by having an edge of the blanket attached to flange 36 (shown in FIG. 1c). The blanket edge is suitably attached to approximately 75% of the flange, with the bottom (chin end) of the mask left open so that there is room to place the mask/hood over the face and head, much like a scarf is placed over one's head and then draped across the neck. In practice, the loose edges of the blanket at the neck and shoulder level would then be gathered and held together in front of the patient with self-sticking tape, hook-and-loop attachment, plastic snap, or other simple mechanism to provide maximum envelopment of the head, neck, shoulders, and upper chest.

The warming hood could be implemented in many different ways. In a preferred implementation, the warming hood's blanket has an outer layer and an inner layer (outer layer 42 shown), with the outer and inner layers heat sealed to each other at their respective perimeters 44. The blanket has at least one access point 46 at which air can be introduced into the blanket between the outer and inner layers; two access points are preferred, located on the right and left side of the patient when the blanket is draped over the head and shoulders, to enable the anesthesiologist to make connection to the hood on whichever side is best for a given operative situation. The inner layer comprises a plurality of micro-perforations (not shown) through which air introduced via the access points is dispersed onto the patient. The outer and inner layers of the blanket are preferably further heat sealed to each other at a plurality of points within the perimeter of said blanket, to form a plurality of linked chambers 48 through which air introduced via the access points is distributed around the interior of the blanket. The outer and inner layers of the warming hood's blanket are suitably thin Mylar sheets in a rectangular configuration of approximately 16×45 inches, though the hood might be made from different materials. For example, one or both layers could be clear medical-grade plastic, or clear Mylar on the ‘up’ side and micro-perforated paper on the ‘down’ (patient) side.

A warming hood as described above could be used with existing Operating Room warm air generators 50 or torso warming devices which force warmed air to the hood via a hose or hoses 52; alternatively, the hood could be provided as a stand-alone device. In this way, the warming hood acts to prevent inadvertent intraoperative hypothermia by heat retention, and to help maintain normal core body temperature.

The present protective mask is preferably intended for a single use. The mask, or components thereof, could be made reusable if a means of sterilizing the components being reused were provided. For example, a protective mask might be provided for which padding 24 is intended to be single-use, but plastic shield 12 is intended to be reused; this would require that a means of sterilizing the plastic shield between uses be provided, along with a means of attaching new padding.

The present protective mask provides a standardized approach to patient facial protection, rather than relying on whatever might be randomly available to an anesthesiologist in the Operating Room. It eliminates surgeon-anesthesiologist confusion regarding facial protection, and provides for predictable, standardized facial protection of patients under general anesthesia in the supine position.

The mask can be advantageously employed wherever facial protection and core body temperature are important, such as low environmental temperature situations, low temperature work environments, search and rescue missions, and outdoor winter activities. The warming hood and blanket can also serve as a “space blanket”, to insulate the individual and reflect heat back to the upper body. In addition to providing facial protection, the present mask can serve as temporary goggles, and aid with heat retention during transport.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

I claim:

1. A protective mask suitable for protecting the face of a patient during surgery, comprising:
a transparent plastic shield sized and shaped to protect the face of a patient when said protective mask is installed on the face of said patient, said shield having a convex topside and an underside and including:
a transverse cutout through said shield which provides access to the patient's nose and mouth; and
a vertical gap in said shield which runs from said transverse cutout to the lower perimeter of said shield;
padding affixed to the perimeter of the underside of said shield which contacts said patient's face and provides a cushion between said shield and said face when said protective mask is installed on the face of said patient, said padding arranged to allow said patient's face to remain substantially visible through said shield when said protective mask is installed on the face of said patient, said padding discontinuous in the area of said vertical gap; and
an attachment mechanism which secures said mask to the face of said patient.

2. The mask of claim 1, wherein said transverse cutout is sized to permit passage of at least two of an endotracheal tube, a laryngeal mask airway tube, and a nasogastric tube.

3. The mask of claim 1, wherein said protective mask is arranged such that, when installed on the face of said patient, said transverse cutout is over said patient's mouth and said vertical gap runs from said transverse cutout to the perimeter of said shield over said patient's chin, such that said transverse cutout and said vertical gap form a T-shaped opening through said shield.

4. The mask of claim 1, wherein said plastic shield, said transverse cutout, said vertical gap and said padding are arranged such that said protective mask can be installed on or removed from the face of said patient without dislodging tubing inserted into said patient's nose or mouth.

5. The mask of claim 1, wherein said plastic shield is a molded plastic shield made from FDA-approved medical grade plastic.

6. The mask of claim 1, wherein said plastic shield is arranged to protect said face, including the eyes, from ultraviolet rays, X-rays, and/or laser light.

7. The mask of claim 1, wherein said padding is pre-cut to provide said discontinuity in the area of said vertical gap.

8. The mask of claim 1, wherein said padding is FDA-approved, hypoallergenic medical grade foam padding.

9. The mask of claim 1, wherein said padding is a flexible plastic bladder containing air or a conformable material.

10. The mask of claim 1, wherein said padding comprises disposable cotton padding.

11. The mask of claim 1, wherein said mask is intended for a single use.

12. The mask of claim 1, wherein said attachment mechanism is affixed to said plastic shield.

13. The mask of claim 1, wherein said attachment mechanism comprises elastic ear loop retainers.

14. The mask of claim 1, wherein said attachment mechanism comprises a behind-head elastic strap.

15. The mask of claim 1, wherein said attachment mechanism comprises a behind-head hook-and-loop strap.

16. The mask of claim 1, wherein said attachment mechanism comprises a four point head strap harness.

17. The mask of claim 1, wherein said attachment mechanism comprises an adhesive.

18. The mask of claim 17, wherein said adhesive is an electron beam cure synthetic polymer.

19. The mask of claim 1, wherein said plastic shield includes a flange extending circumferentially from the perimeter of said shield, said flange substantially forming a right angle with the topside of said shield.

20. The mask of claim 19, wherein said padding comprises first and second pieces of foam padding which are sandwiched on and affixed to either side of said flange.

21. The mask of claim 20, wherein said attachment mechanism comprises elastic ear loop retainers, each of which passes through said sandwiched foam padding and is affixed to said plastic shield.

22. The mask of claim 1, further comprising a warming hood arranged to be coupled to said protective mask and which covers the head, neck and shoulders when so coupled.

23. The mask of claim 22, wherein said warming hood comprises a blanket having an outer layer and an inner layer, said outer and inner layers heat sealed to each other at their respective perimeters, said blanket having at least one access point at which air can be introduced into the blanket between the outer and inner layers, said inner layer comprising a plurality of micro-perforations through which air introduced via said access points is dispersed onto said patient.

24. The mask of claim 23, wherein said outer and inner layers are Mylar sheets.

25. The mask of claim 23, wherein said outer and inner layers are further heat sealed to each other at a plurality of points within the perimeter of said blanket.

26. The mask of claim 25, wherein said plurality of heat sealed points within the perimeter of said blanket form a plurality of linked chambers through which air introduced via said access points is distributed around the interior of said blanket.

27. The mask of claim 1, wherein said transverse cutout is approximately 4 inches wide by 1 inch high, and said vertical gap is approximately 0.75 inches wide by 3 inches high.

28. A method of protecting the face of a patient during surgery, comprising:
placing the patient in the supine position;
inserting tubing as necessary into the patient's nose and mouth;
installing a protective mask over the patient's face, said protective mask comprising:
a transparent plastic shield sized and shaped to protect the face of a patient when said protective mask is installed on the patient's face, said shield having a convex topside and an underside and including:
a transverse cutout through said shield which provides access to the patient's nose and mouth; and
a vertical gap in said shield which runs from said transverse cutout to the perimeter of said shield;
padding affixed to the perimeter of the underside of said shield which contacts said patient's face and provides a cushion between said shield and said face when said protective mask is installed on the face of said patient, said padding arranged to allow said patient's face to remain substantially visible through said shield when said protective mask is installed on the face of said patient, said padding discontinuous in the area of said vertical gap; and
an attachment mechanism which secures said shield and padding to said patient's face;
said installing step comprising manipulating said mask such that said tubing, if any, passes through said vertical gap and becomes located within said transverse cutout; and
attaching said protective mask to said patient's face using said attachment mechanism.

29. The method of claim 28, wherein said attachment mechanism comprises elastic ear loop retainers, said step of attaching said protective mask comprising installing said elastic ear loop retainers around the patient's ears.

30. The method of claim 28, further comprising coupling a warming hood to said protective mask, said warming hood arranged to cover the head, neck and shoulders when so coupled.

31. The method of claim 30, wherein said warming hood comprises a blanket having an outer layer and an inner layer, said outer and inner layers heat sealed to each other at their respective perimeters, said blanket having at least one access point at which air can be introduced into the blanket between the outer and inner layers, said inner layer comprising a plurality of micro-perforations through which air introduced via said access points is dispersed onto said patient;
said method further comprising:
providing a warm air generator capable of providing a flow of warmed air; and
coupling the output of said warm air generator to said warming hood.
